(19) United States
(12) Patent Application Publication
(10) Pub. No.: US 2019/0083671 A1
(43) Pub. Date: Mar. 21, 2019

(54) HUMIDIFIER WITH AROMATHERAPY TRAY

(71) Applicant: Great Innovations, LLC, Miramar, FL (US)

(72) Inventor: Joseph A. McDonnell, Weston, FL (US)

(21) Appl. No.: 16/137,285

(22) Filed: Sep. 20, 2018

Related U.S. Application Data
(60) Provisional application No. 62/561,192, filed on Sep. 20, 2017.

(51) Int. Cl. A61L 9/14; A61M 21/00; F24F 6/12

(52) U.S. Cl. CPC A61L 9/14; A61M 21/00; A61L 2209/111; A61M 2021/0016; F24F 6/12

(57) ABSTRACT

The humidifier disclosed herein may include a trap door for housing one or more chemically impregnated filter papers, referred to herein as “aroma pads,” containing aromatherapy oils and/or fragrances. In use, air is impregnated with the aromatherapy oils and/or fragrances, and the infused air is mixed with mist produced by the humidifier and disbursed into area served by the humidifier. The aroma pads may be housed in slots formed in the trap door, or, alternatively, in a tray inside an aroma compartment within the humidifier.

### CROSS REFERENCE TO RELATED APPLICATION

This is a non-provisional patent application of U.S. Provisional Patent Application No. 62/561,192, filed Sep. 20, 2017, the entire contents of which is hereby incorporated by reference.

### FIELD OF THE DISCLOSURE

The present disclosure generally relates to room humidifiers. In particular, the disclosure relates to a humidifier that includes an integrated aromatherapy tray.

### BACKGROUND OF THE DISCLOSURE

It is known that aromatherapy may use a variety of different natural oils to improve physical and psychological health of an individual. These oils may typically be extracted from bark, stems, leaves, flowers, roots or other plant parts. When these oils are used properly, they may emit an aroma that can stimulate brain function to boost a healing process and improve relaxation. The popularity of aromatherapy has been growing quickly and more people use it as a form of alternative medicine to promote enhanced cognitive function, improved mood and pain relief. A humidifier may serve as one of the ways to ensure a user to enjoy the benefits of aromatherapy when the humidifier may be configured to simultaneously spread the fragrance of natural oils and provide humidity to a room or a given area near the humidifier.

Accordingly, it is desirable to provide a humidifier that includes an integrated aromatherapy tray.

### SUMMARY OF THE DISCLOSURE

The present disclosure relates to a humidifier having a base portion configured to produce mist and an aromatherapy tray integrated with the base portion. The aromatherapy tray is configured to receive and retain aromatherapy oils or fragrance, and the base portion is configured to infuse the mist with the aromatherapy oils or fragrance to created scented mist and direct the scented mist through an opening of the humidifier at a selected air velocity.

### BRIEF DESCRIPTION OF THE DRAWINGS

FIG. 1 is a perspective view of the preferred embodiment of the humidifier of the present disclosure;

FIG. 2 is a perspective view of the humidifier of FIG. 1 showing the trap door in the humidifier into which aroma pads can be inserted;

FIG. 3 is an enlarged view of the trap door of FIG. 2 showing dual aroma pads inserted therein;

FIG. 4 is a bottom view of the humidifier of FIG. 1 showing the aroma base;

FIG. 5 illustrates rear and cutaway views showing the trap door that holds the aroma pads;

FIG. 6 is a perspective view illustrating the inside of the trap door that holds aroma pads;

FIG. 7 is a cutaway view illustrating airflow through the aroma compartment of the humidifier;

FIG. 8 is a cutaway view illustrating airflow through the humidifier when an aroma tray is added to hold an additional aroma pad; and

FIG. 9 is a cutaway view illustrating the airflow through the humidifier without a trap door and without any aroma pads.

### DETAILED DESCRIPTION OF THE DISCLOSURE

Specific structural and functional details disclosed herein are merely representative for purposes of describing example embodiments of the present disclosure. This disclosure may, however, be embodied in many alternate forms and should not be construed as limited to only the embodiments set forth herein.

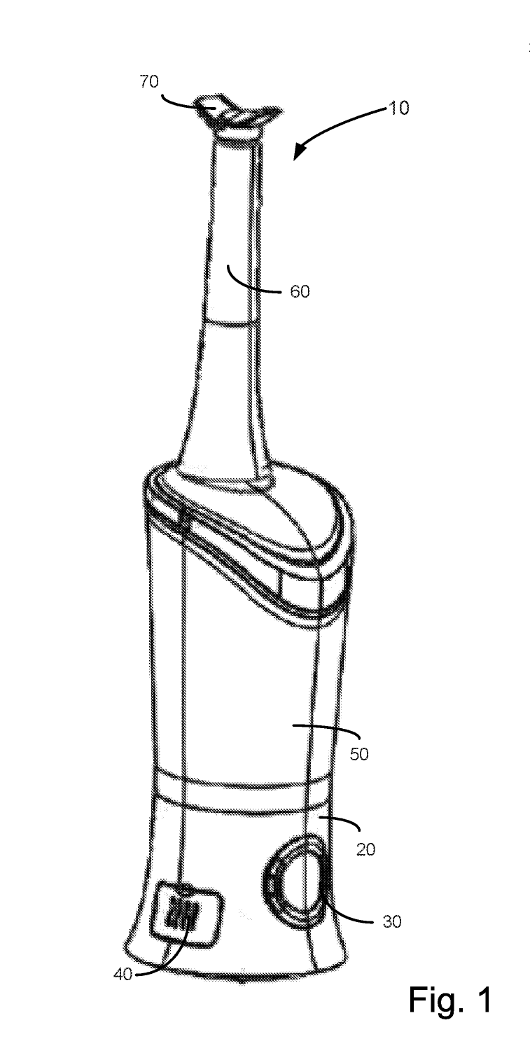

As shown in FIG. 1, a humidifier 10 with an aromatherapy tray, in accordance with aspects of the present disclosure, may be used on, e.g., an elevated surface, such as a tabletop or desk, or on the floor. Humidifier 10 may include a base portion 20, a control panel 30, an aromatherapy tray 40, a reservoir 50, a detachable extending portion 60, and a nozzle 70. The control panel 30 may be included in the reservoir 50 or be a separate unit, such as a wired or wireless remote control. A detachable extending portion 60 may allow the humidifier 10 to be converted from a tabletop or desktop humidifier into a floor standing humidifier. As a result, the detachable extending portion 60 may allow the mist vapor produced by the humidifier 10 to be released at an increased height that puts the moisturizing mist where a user needs it for use throughout a room.

The base portion 20 may be configured to house at least a first set of electronics used by the humidifier 10 to produce mist and a second set of electronics used by the control panel 30 to control the production of mist. The base portion 20 may be connected to a power source (not shown) to provide power to various electronic components of the humidifier 10.

According to one embodiment, the base portion 20 may be configured to produce mist via ultrasonic nebulization, but any other known mist producing method may also be used. The base portion 20 may house a mist producing unit, a humidistat for determining the ambient humidity in the surrounding environment, a water sensor for determining whether water is present in the reservoir 50 of the humidifier 10, an air outlet for directing the flow of mist from the base portion 20, and/or a speaker for providing audible information. The air outlet may include a fan that is configured to control the flow of mist from the base portion 20 and through the reservoir 50.

The electronics housed in the base portion 20 may include a processor and a memory. The processor and memory may be electronically connected to the mist producing unit, the control panel 30, humidistat, water sensor, speaker, fan and other components. The processor and memory may be configured to control the operations of the humidifier 10 and execute various functions thereof.

The control panel 30 may include a display for displaying information about the humidifier 10 and a plurality of buttons for operating the humidifier 10. For example, a user may use one of the plurality of buttons to turn on or turn off the humidifier 10, delay the start of the humidifier 10, automatically turn off the humidifier 10, control the output level of mist of the humidifier 10, set a relative humidity level desired, and dim the display. In one aspect, a mist output function of the control panel 30 may allow programming of the output level of mist of the mist producing unit. By using the buttons on the control panel 30, a user may increase and decrease the amount of mist produced by the humidifier 10. The air outlet may increase the air flow from the base portion as the desired output level of mist increases. The air flow from air outlet allows the mist produced by the base portion 20 to be direct through the reservoir 50.

The reservoir 50 may be configured to supply and hold water used by the base portion 20 to produce mist, and the reservoir 50 may be detachable from the base portion 20. In one aspect, the reservoir 50 may include a tank, a tank cap, and a pass-through connection. Specifically, the tank of reservoir 50 may hold the water for use by the base portion 20 to produce mist. The tank cap may be removable from the tank to allow for easy filling of water into the tank. Tank cap may also be used in supplying base portion 20 with water when reservoir 50 is connected to base portion 20. A detachable filter may also be removably connected to tank cap to filter the water used by the base portion 20 in production of mist. The detachable filter may be a permanent filter or a replaceable filter. The detachable filter may also be an antibacterial filter, a demineralization, an antibacterial and demineralization filter, or any other known water type filters. The pass-through connection of the reservoir 50 may be configured to direct the mist produced by the base portion 20 to flow through the reservoir 50 and exit an opening on the top surface of the reservoir 50. A fan provided in air outlet may be configured to direct the flow of mist produced in the base portion 20 through the pass-through connection. As shown in FIG. 1, when the humidifier 10 is in an extended position using the detachable extending portion 60, the flow of mist may flow through the extending portion 60 and exit the nozzle 70 at an elevated height. Nozzle 70 may be removable and connected directly to the opening where the detachable extending portion 60 connects to the reservoir 50. As such, the humidifier 10 may have at least two operation modes providing mist vapor at different locations and heights. Accordingly, the processor and memory of the base portion 20 may be configured to detect the operation mode of the humidifier 10 and adjust the fan output power to propel the generated mist through the pass-through connection and exit the nozzle 70. Further, once the nozzle 70 is detachably connected to the opening on the top surface of the reservoir 50 in a first operation mode or to the extending portion in the second operation mode of the humidifier 10, a plurality of movable nozzle openings may be configured to direct the flow of mist in a plurality of directions. That is, the moveable nozzle openings may be configured to direct mist in substantially the same direction, or in a plurality of different directions. As a result, different mist dispersion areas may be achieved.

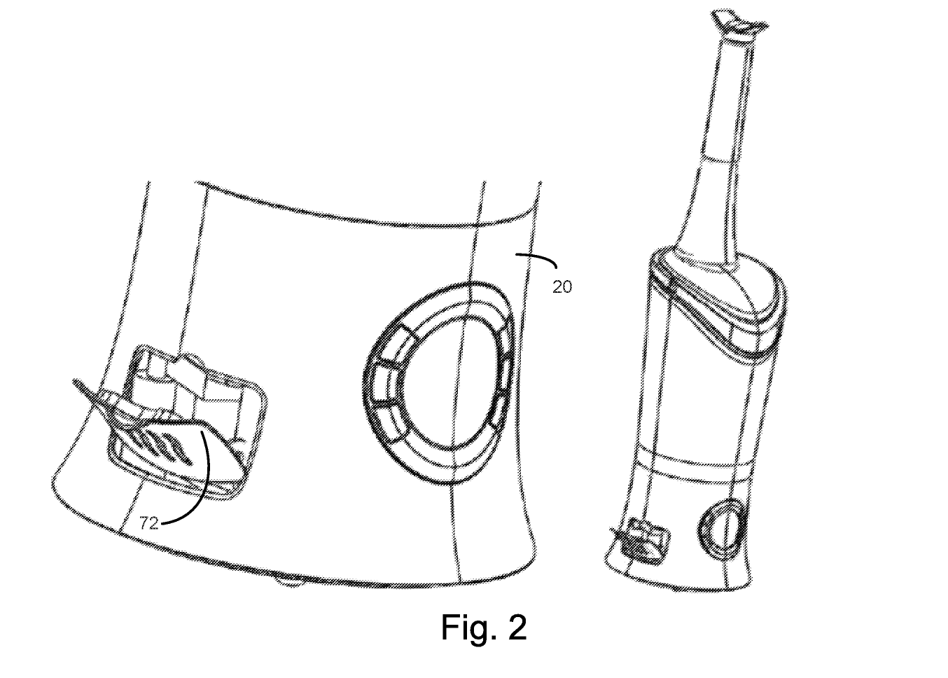

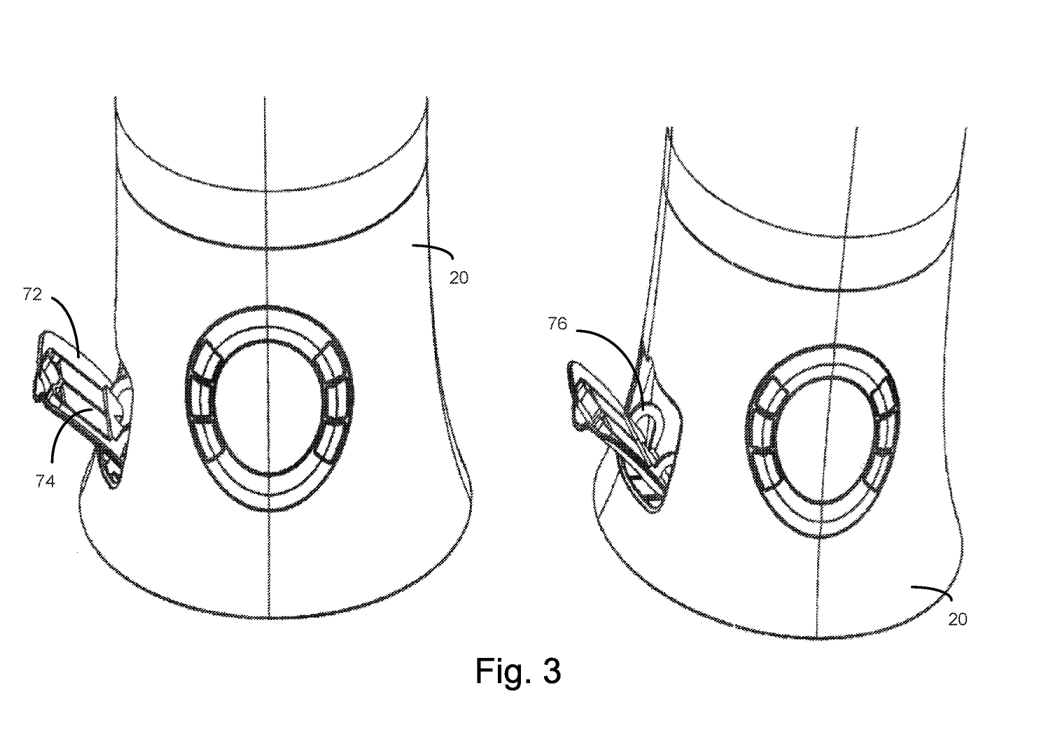

As shown in FIGS. 1-3, according to aspects of the present disclosure, humidifier 10 may include the aromatherapy tray 40 integrated on the base portion 20 for facilitating aromatherapy. In one embodiment, a trap door 72 with a vented tray 74 may be configured to hold chemically impregnated filter paper, which is referred to as an “aroma pad” containing aromatherapy oils/fragrance. Trap door 72 is pivotally attached to base portion 20 of humidifier 10 using hinged members 76, flush with the outer surface of the base portion 20 when closed. The aroma pad may be placed in the vented tray 74 or a location easily accessible through the trap door 72 of the humidifier 10, such that a used aroma pad may be easily replaced or refilled when the scent has been used up. The vented tray 74 may also accommodate more than one aroma pad, in order to provide a more intense aroma, or a composite aroma. For example, as shown in FIG. 3, dual aroma pads may be inserted into the vented tray 74.

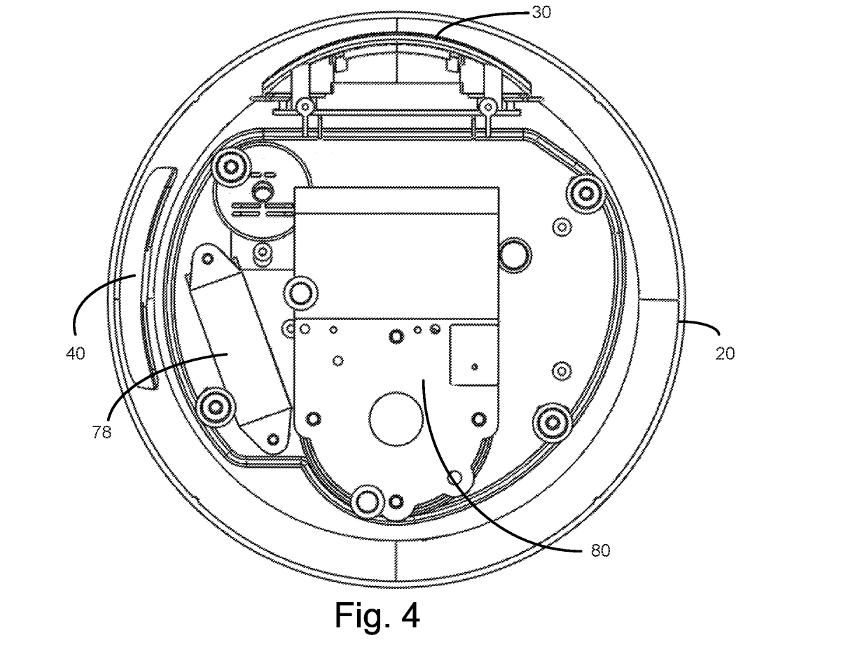

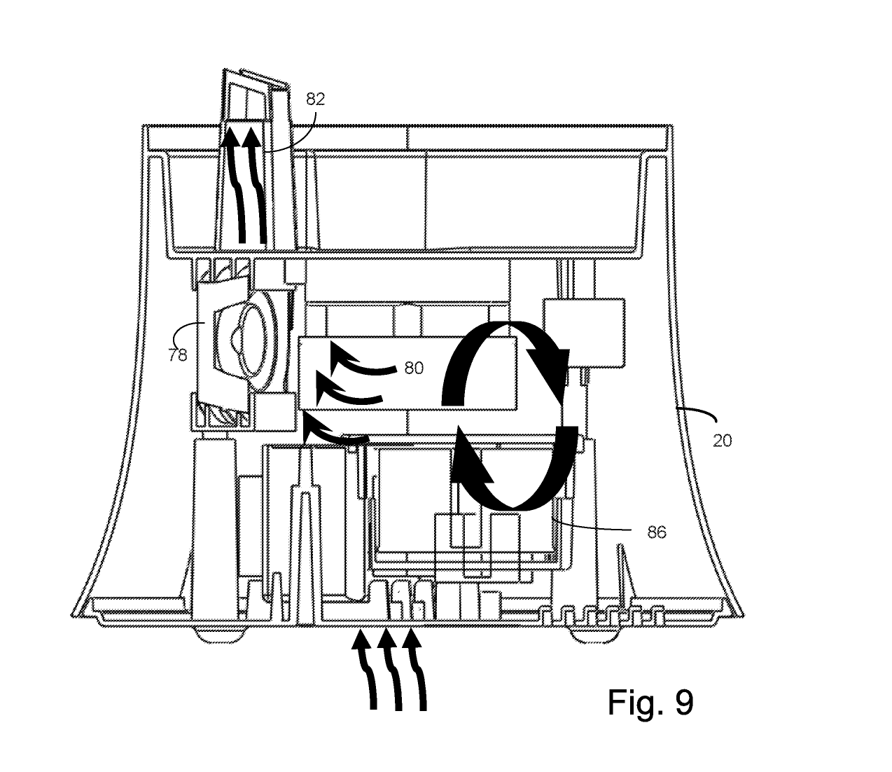

FIG. 4 is a bottom view of humidifier 10 showing the aroma base. More specifically, a fan 78 may be positioned inside the base portion 20 and in proximity to the compartment door of the aromatherapy tray 40. In accordance with aspects of the present disclosure, humidifier 10 may be an ultrasonic humidifier using a submerged, vibrating metal diaphragm or ultrasonic board of an ultrasonic nebulizer module 80 in the base portion 20 to impart mechanical energy to the water inside the reservoir 50. The use of ultrasonic energy results in the formation of an atomized cool mist above the surface of the reservoir 50. For example, a transducer may be used to transform electrical energy into mechanical energy for generating the cool mist. The transducer may include a nebulizer which vibrates quietly at greater than at least, e.g., 1.5 million times per second in order to convert water into a fine, cool mist. As described above, detachable reservoir 50 may be configured to supply and hold water used by the base portion 20, such that the nebulizer 80 of the base portion 20 may continuously churn the water into a fine, cool mist. FIG. 9 generally illustrates the airflow through an ultrasonic humidifier without a trap door and without any aroma pads. An embodiment of such ultrasonic humidifier without a trap door and without any aroma pads is disclosed in U.S. Pat. No. 9,752,790, the entire contents of which is hereby incorporated by reference.

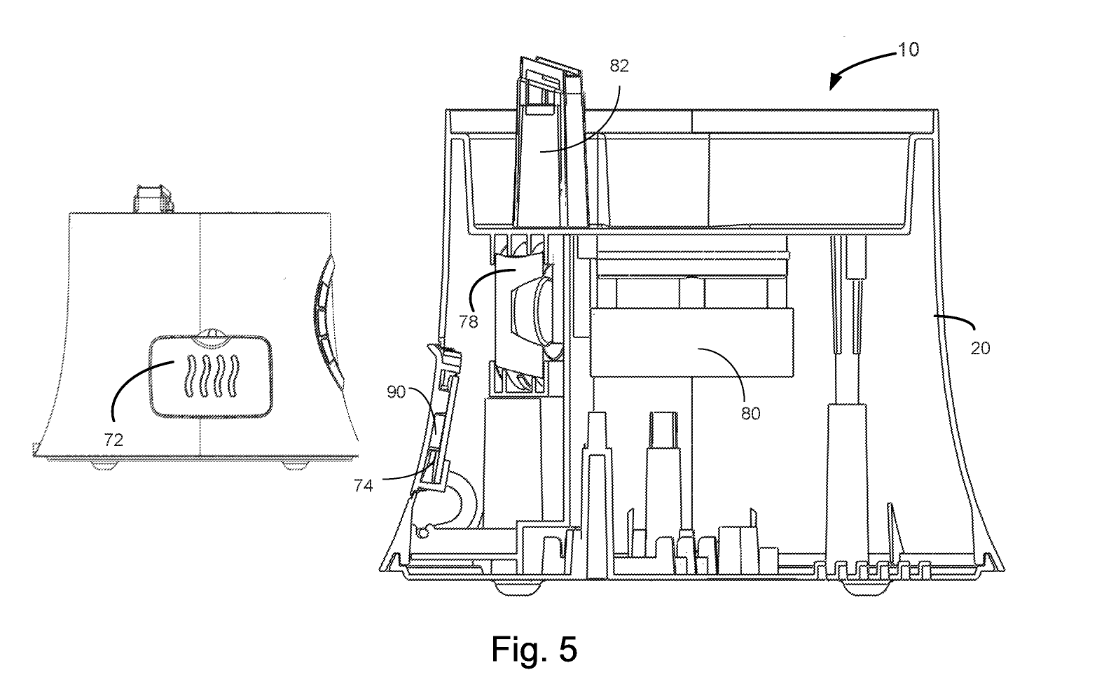

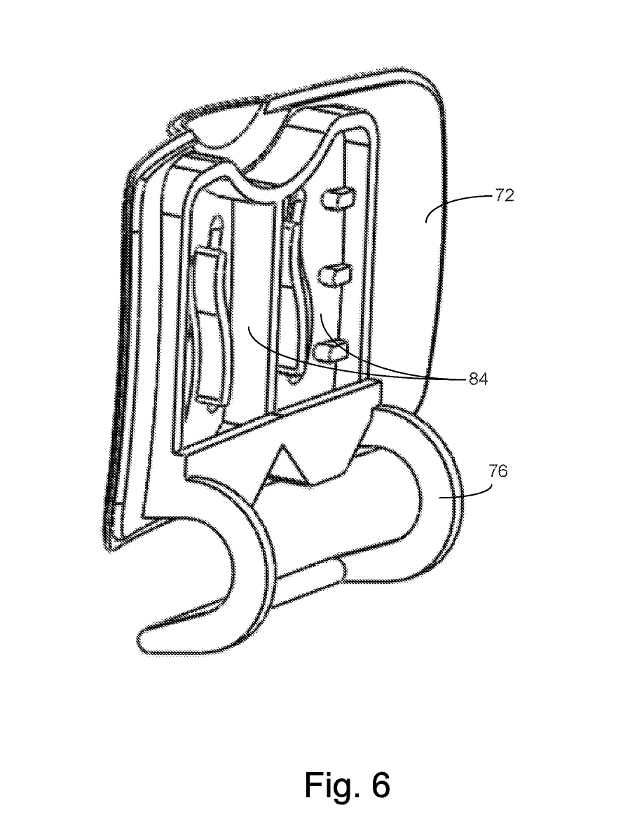

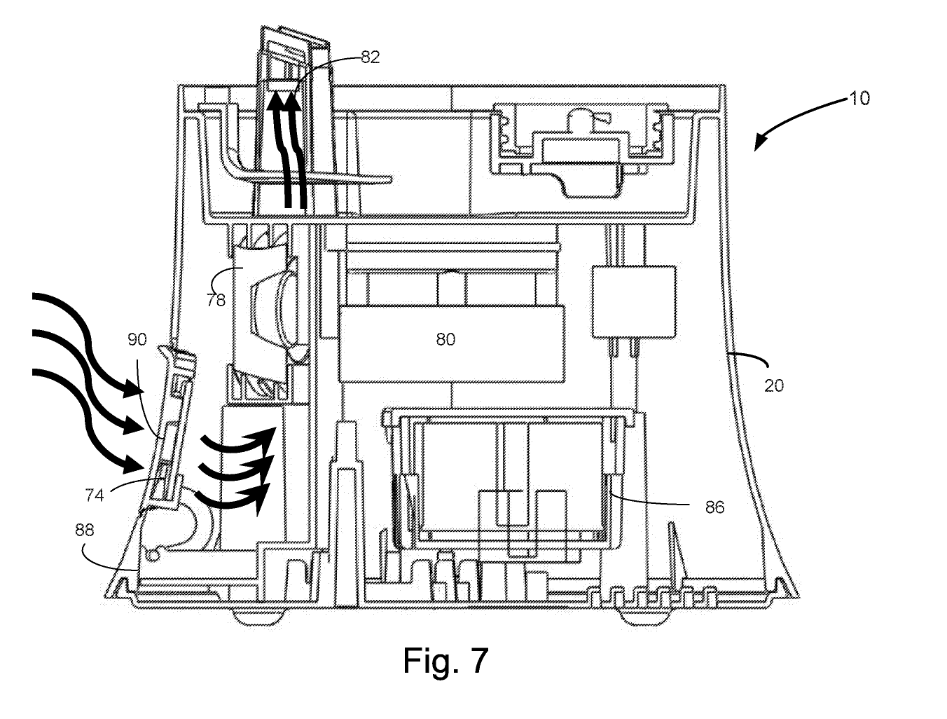

Referring to FIGS. 5-8, the base portion 20 of the humidifier 10 may include components that form an airflow channel for pulling exterior air through the trap door 72 to fan 78 within the humidifier 10. The base portion 20 may also include electronics to control power supply 86 and fan 78 to create the necessary air velocity to push the mist vapor to the surrounding external environment of humidifier 10 through, e.g., mist outlet 82, the pass-through connection, the detachable extending portion 60, and/or the nozzle 70. For example, as shown in FIGS. 5 and 6, at least one aroma pad 90 may be inserted into the aroma tray 74 of one of the aroma pad compartments 84 that is exposed when the trap door 72 is opened. Subsequently, the trap door 72 may be closed and held in place by a tab 88 inside the humidifier 10. The fan may be controlled to pull exterior air past the aroma pad 90 inserted in the vented tray 74 and infuse the air with the mist vapor created by the humidifier 10. The infused air including the scented mist may then be distributed into the surrounding environment.

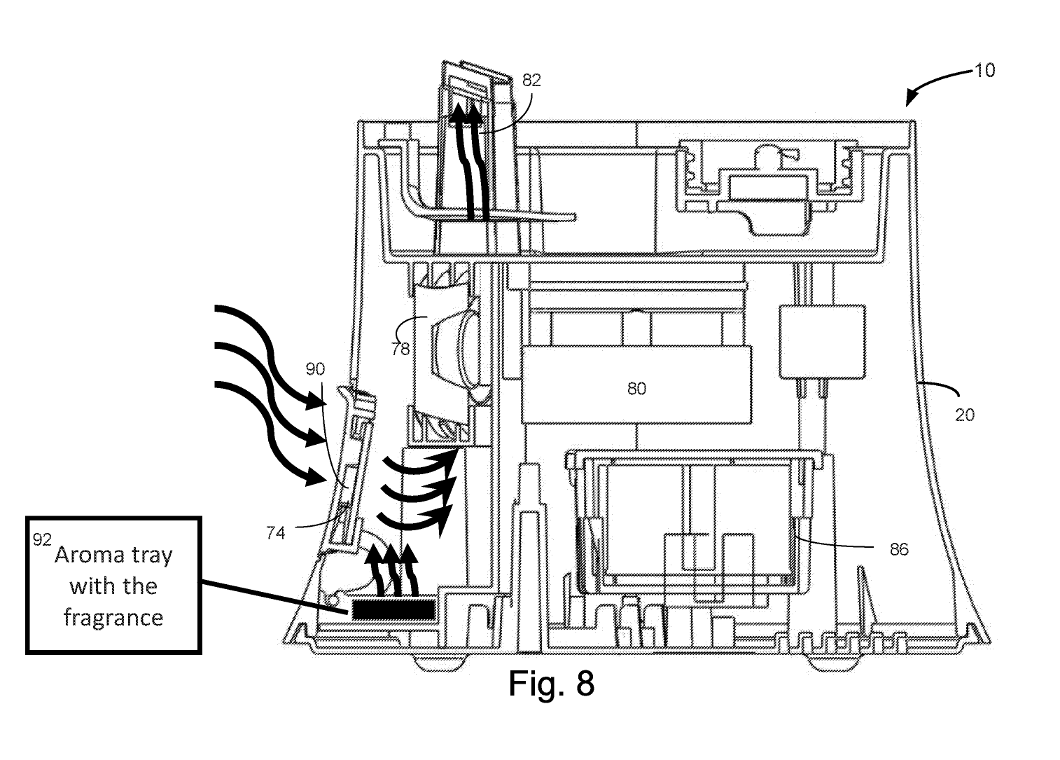

In accordance with aspects of the present disclosure, as shown in FIG. 8, a plurality of aroma tray 92 may be placed inside the base portion 20. As a result, the exterior air drawn into the base portion 20 may be initially aromatized by the aroma pad 90, and the aromatherapy oils or fragrance provided by aroma tray 92 may further aromatize the air before it is infused with the mist produced by the nebulizer 80.

The many features and advantages of the disclosure are apparent from the detailed specification, and thus, it is intended by the appended claims to cover all such features and advantages of the disclosure which fall within the true spirit and scope of the disclosure. Further, since numerous modifications and variations will readily occur to those skilled in the art, it is not desired to limit the disclosure to the exact construction and operation illustrated and described, and accordingly, all suitable modifications and equivalents may be resorted to, falling within the scope of the disclosure.

1. A humidifier, comprising:
a base portion configured to produce mist; and
an aromatherapy tray integrated with the base portion, the aromatherapy tray being configured to receive and retain aromatherapy oils or fragrance,
wherein the base portion is configured to infuse the mist with the aromatherapy oils or fragrance to created scented mist and direct the scented mist through an opening of the humidifier at a selected air velocity.

2. The humidifier of claim 1, wherein the base portion is configured to produce mist by ultrasonic nebulization.

3. The humidifier of claim 1, wherein the aromatherapy tray is integrated with the base portion via a trap door that is flush with the base portion when the trap door is closed.

4. The humidifier of claim 1, wherein the aromatherapy tray includes a compartment for receiving and retaining one or more aroma pads containing the aromatherapy oils or fragrance.

5. The humidifier of claim 4, wherein the base portion comprises a fan configured to draw ambient air through the one or more aroma pads to infuse with the mist produced.

6. The humidifier of claim 1, further comprising a plurality of aroma trays inside the base portion for receiving and retaining aromatherapy oils or fragrance.

7. The humidifier of claim 1, wherein the base portion is configured to detect an operation mode of the humidifier and determine an air velocity of the scented mist based at least on the detected operation mode.
